(19) United States
(12) Patent Application Publication
(10) Pub. No.: US 2012/0239061 A1
(43) Pub. Date: Sep. 20, 2012

(54) ENDOSCOPIC FULL THICKNESS GASTRIC REDUCTION APPARATUS AND METHOD

(76) Inventor: Sandip V. Mathur, Abilene, TX (US)

(21) Appl. No.: 13/048,229

(22) Filed: Mar. 15, 2011

(51) Int. Cl. A61B 17/10; A61F 2/00

(52) U.S. Cl. 606/140; 600/37

(57) ABSTRACT

A device, adapted to receive one or more gastric bands and adapted to be coupled to a surgical instrument such as an endoscope, comprises a tubular main body and a cap comprising an opening through which a guidewire may be threaded. A triggering wire may be detachably coupled to one or more bands to permit remote removal of said bands. The bands may be improved bands comprising gripping points, an inner reinforcement ring, or an expanded surface area portion.

A method for performing band ligation comprises the steps of: providing a device comprising a main body portion, a removably coupled cap, one or more gastric bands, and a band triggering wire; introducing the device through the mouth into the stomach; dislodging the cap; targeting and drying specific locations of the gastric wall; withdrawing a portion of the gastric wall into the device; and displacing one or more of the bands such that the band circumscribes said wall portion; and the administration of medication to prevent gastric contractions.

### BACKGROUND OF THE INVENTION

1. Field of the Invention

The present invention relates generally to a surgical device and specifically to an endoscope mounted apparatus and method that permits entrapment and destruction of stomach tissue.

2. Description of the Prior Art

Obesity, a medical condition that results from the accumulation of excess body fat, is a major medical challenge in many parts of the world and is considered one of the leading preventable causes of death. The condition contributes directly to numerous adverse health conditions such as heart disease, diabetes and certain cancers. Such conditions, and others caused or exacerbated by obesity, can lead to reduced life expectancy and serious quality of life issues.

Obesity is commonly caused by a combination of excessive caloric intake and insufficient physical activity. Consequently, the primary traditional treatment for obesity is attempting to decrease caloric intake while at the same time increasing the number of calories burned—i.e. diet and exercise. In many patients, diet and exercise regimens are unsuccessful over the long term. In such cases, some physicians recommend certain medications intended to reduce the patient's appetite and, thus, caloric intake.

In more severe cases of obesity, such as when the patient is 80 to 100 pounds overweight, various surgical techniques have been utilized, including: adjustable gastric band (AGB). Roux-en-Y gastric bypass (RYGB), biliopancreatic diversion with a duodenal switch (BPD-DS), and vertical sleeve gastrectomy (VSG).

Because obese patients are generally poor surgical candidates, it is often desirable to perform minimally invasive procedures through an endoscope. Endoscopic procedures do not require the body cavities to be cut open. They require less anesthesia and take less time than conventional surgery. Hence, endoscopic therapy is often preferred in the obese.

Currently, typical endoscopic techniques allow only the innermost lining of the stomach to be grasped and manipulated. As stomach bleeding occurs from the innermost lining of the stomach, it is amenable to endoscopic therapy. One well-established procedure for control of such bleeding is Band Ligation which involves ligating or tying-off portions of the innermost lining of walls of the digestive tract. In this procedure, a medical instrument is introduced through the patient's mouth into the digestive tract. This procedure permits the physician to control bleeding from the innermost layer of the wall of the digestive tract and is routinely conducted through the use of conventional instruments and techniques. It is now proposed that the technique be expanded to involve the ligation of all four layers of the stomach wall with the purpose of reducing the volume of the stomach and permitting early satiety.

Ligation of the full thickness of the wall entails dealing with a greater amount of tissue and, therefore, requires a much larger device than is currently available. Conventional and commercially available instruments do not presently permit the surgeon to grasp the full thickness of the gastric wall as well as some part of the external fatty tissue that surrounds it. At present, a surgeon who attempts to introduce a large caliber device through the oral cavity and into the stomach is likely to encounter significant resistance and difficulty in maneuvering such a device through the esophagus and into the stomach. Moreover, with conventional technology, such an attempt could cause severe trauma and even perforation of the esophagus.

A novel method of targeting the fundus (roof) and/or lateral capacitance areas of the stomach is presented which hastens the sense of early satiety but preserves the normal stomach functions related to the storage and grinding of food. The method comprises drying a targeted area with a jet of air transmitted through the endoscope, using one or more conventional or new bands, and the use of medication to prevent gastric contractions in the post-operative phase in order to prevent band dislodgement.

The present invention permits a physician to engage and trap larger amounts of stomach tissue than is presently possible through conventional laparoscopy or endoscopy. To overcome the problems associated with large caliber devices, the invention provides a cap which may be removed inside the stomach. The cap permits the passage of a much larger instrument. An improved band structure, banding technique and, as discussed above, a new method of involving targeted application is provided. Currently, band ligation instruments are inadequate for ligating full thickness tissue, have no cap and encounter resistance upon passage through the esophagus.

The device and method of the present invention allow physicians to help their patients control weight by use of an instrument and procedure that is inexpensive, rapid, endoscopic rather than surgical, simple to administer, long-lasting, and retains the storage and grinding functions of the stomach.

### SUMMARY OF THE INVENTION

A device is provided comprising a tubular main body or barrel with a central cavity and a conical, spherical or tubular cap. Disposed around a perimeter of the device body are one or more elastic gastric bands. At a distal end of the device is the cap which is comprised of an opening through which a guidewire may be threaded. In other embodiments, the device also has a triggering wire detachably coupled to each of the bands so that a surgeon can remotely remove the bands from the device body perimeter by movement of the triggering wire. The device is coupled at a proximal end to a distal end of an endoscope.

In operation, the surgeon endoscopically inserts the guidewire through a patient's mouth, esophageal opening, and into the patient's stomach, and threads the device mounted on the endoscope over the same external guidewire end towards the stomach. Once the device is introduced into the stomach, the surgeon may remotely remove the cap with endoscopic forceps. The surgeon, in one embodiment of the present invention, then targets a site in the fundus (roof) of the stomach. The surgeon prepares the site for band placement by first drying the site with a jet of air introduced through the endoscope. This activity dries the slick mucus that could otherwise permit the band to slide off, particularly during a later stomach contraction. The surgeon, using forceps may then grasp a portion of the stomach lining to form a gathered portion and retract this portion into the cavity of the device. The surgeon, may then, using the triggering wire, displace one or more of the bands so that it or they contract(s) around the gathered portion. Additional portions of the stomach wall may be banded in a like or similar manner. Upon conclusion of the procedure, the surgeon may grasp the cap, with the forceps and retract the instrument.

Also provided is a method for performing band ligation comprising the steps of: providing a device comprising a main body portion, a cap, one or more gastric bands, and a band triggering wire, the cap being removably coupled to said main body portion; introducing the device through the mouth and esophageal opening and into the stomach, the stomach having a gastric wall; dislodging the cap from the main body portion; targeting and drying the roof of the stomach; withdrawing a portion of the gastric wall into the device to form a gathered portion; and displacing one or more of the bands such that the band circumscribes said gathered portion.

Also provided is a method for performing band ligation comprising the steps of: providing a device comprising a main body portion, a tapered distal portion, one or more gastric bands, and a band triggering wire, said tapered distal portion being removably coupled to said main body portion; providing a guidewire having first and second ends; inserting the guidewire first end through a patient's mouth, through an esophageal opening, and into a patient's stomach, the stomach having a gastric wall; introducing the device over the guidewire second end through the mouth and esophageal opening and into the stomach; removal of the guidewire; dislodging the distal tapered portion from said main body portion; withdrawing a full thickness of the gastric wall into the device; and displacing one or more of said bands such that said band circumscribes a portion of said wall.

In one embodiment, bands of improved structure are used.

In one embodiment, the cap is constructed of an inert or digestible material such as cellulose or vegetable fiber and does not have to be retrieved. In another embodiment, the cap is retrievable. In another embodiment, the cap is transparent.

In one embodiment of the present invention, the cap is conical. In another embodiment the cap is spherical. In another embodiment, the cap is elongated. In another embodiment the cap is beveled. In still another embodiment, the cap is eccentric or angulated.

In one embodiment of the present invention, the cap is perforated to permit the passage of a guidewire.

In one embodiment of the present invention, the base of the cap has a through opening or channel structured and arranged to permit the passage of one or more triggering wires.

In one embodiment of the present invention, the distal end of the barrel has a through opening or channel structured and arranged to permit the passage of one or more triggering wires.

In one embodiment, the cap and barrel distal end triggering wire channels are capable of being arranged to form a combined triggering wire channel.

In one embodiment of the present invention, the cap is attached to the barrel with a tether.

In one embodiment of the present invention the cap, is connected externally by means of a small diameter hollow tube through which water may be flushed onto the cap's surface to remove debris and lubricate the tip of the assembly which tube may be used to extricate the cap after the procedure.

In one embodiment of the present invention, a distal end of the barrel is vertical. In another embodiment, the distal end has an angular configuration.

In one embodiment of the present invention, the barrel comprises a balloon which may be inflated and deflated, such that the device may be used without a cap or to displace a cap.

In one embodiment of the present invention, the barrel's proximal end has an angular configuration.

In one embodiment, the device is introduced over a guidewire passing through a perforation in the cap. In another embodiment, the wire passes through a hook or channel external to the barrel.

In one embodiment, the device may be introduced under direct vision through a transparent cap.

In one embodiment, the distal barrel opening is on a barrel side wall.

In one embodiment, tissue is suctioned into the apparatus using vacuum suction.

In one embodiment, after band ligation of the stomach tissue, the tissue is injected with a destructive agent such as absolute alcohol. In another embodiment, the tissue is thermally destroyed.

In one embodiment, the forceps are snare forceps.

In one embodiment, a greater number of bands is placed in the fundus with fewer bands placed in a lower position in an inverted triangle or other pattern.

In one embodiment, the method is used to reduce the size of a hiatal hernia.

In one embodiment, the method is used to treat hyperacidity. In another embodiment, the method is used to treat acid reflux and Barrett's esophagitis.

In one embodiment, the grasped tissue is rotated clockwise or counter-clockwise as it is withdrawn into the chamber before band ligation.

In one embodiment, the bands are at least partially reinforced with an inner reinforcement ring.

In other embodiments, the bands may be irregular, internally serrated or V shaped or are larger and/or flatter, or have a mesh or perforated structure to enhance tissue grip and adherence.

In one embodiment, two or more bands may be placed on the withdrawn tissue.

In one embodiment, portions of the stomach are air dried.

In one embodiment, gastric contractions are prevented after placement of the bands by the use of intravenous medications.

### BRIEF DESCRIPTION OF THE DRAWINGS

FIG. 1 is a side elevation view of the apparatus coupled to an endoscope in accordance with a preferred embodiment.

FIG. 2 is a front view of the apparatus coupled to an endoscope in accordance with a preferred embodiment.

FIG. 3 is a rear view of the apparatus coupled to an endoscope in accordance with a preferred embodiment.

FIG. 4 is a side view of a patient and device showing the device before it is inserted into the patient and with the guidewire inserted into the patient's stomach.

FIG. 5 is a side view of the device with forceps extended in position to grasp a portion of the gastric lining.

FIG. 6 is a side view of the device of FIG. 5 with a portion of the gastric lining retracted within the device.

FIG. 7 is a side view of the device of FIGS. 5 & 6 with the gastric lining in the retracted position after a band has been deployed.

FIG. 8 is a side view of the device of FIGS. 5-7 with the banded gastric lining outside the device.

FIG. 8b is a side view of the device of FIGS. 5, 6 and 7 with two bands placed on the gastric lining, shown outside the device.

FIG. 8c is a top side plan view of an improved gastric band in accordance with a preferred embodiment.

FIG. 8d is a top side plan view of an improved gastric band in accordance with another embodiment.

FIG. 8e is an isometric view of an improved gastric band in accordance with another embodiment.

FIG. 8f is an isometric view of an improved gastric band in accordance with still another embodiment.

FIG. 8g is a side elevation view of the gastric bands of FIGS. 8e and 8f.

FIG. 8h is a top side elevation view of the gastric bands of FIGS. 8e and 8f.

FIG. 8i is an isometric view of the gastric band in accordance with another embodiment.

FIG. 8j is an elevation view of the gastric band of FIG. 8i in a contracted and deployed position around a portion of gastric tissue.

FIG. 8k is an isometric view of the gastric band in accordance with another embodiment.

FIG. 8L is an elevation view of the gastric band of FIG. 8k in a contracted and deployed position around a portion of gastric tissue.

FIG. 9 is a side view of the device with the cap in a retracted position.

FIG. 10a is a side view of the device in accordance with another embodiment.

FIG. 10b is a side view of the device in accordance with another embodiment.

FIG. 10c is a side view of the device in accordance with another embodiment.

FIG. 10d is a side view of the device in accordance with another embodiment.

FIG. 10e is a side view of the device in accordance with another embodiment.

FIG. 10f is a side view of the device in accordance with another embodiment.

FIG. 10g is a side view of the device in accordance with another embodiment.

FIG. 11 is a side view of the device with a guidewire extending through a cap perforation in accordance with another embodiment.

FIG. 12 is a side view of the device in accordance with another embodiment.

FIG. 13 is a side view of the device in accordance with another embodiment.

FIG. 14 is a side view of the device in accordance with another embodiment.

FIG. 15 is a side view of the device in accordance with another embodiment.

FIG. 16 is a side view of the device in accordance with another embodiment.

FIG. 17 is a side view of the device in accordance with another embodiment.

FIG. 18 is a side view of the device showing snare entrapment and injection/cauterization of the captured gastric lining.

FIG. 19 is a diagram showing the placement of bands in another aspect of the invention.

FIG. 20 is a diagram showing the placement of bands in another aspect of the invention.

### DESCRIPTION OF THE PREFERRED EMBODIMENT

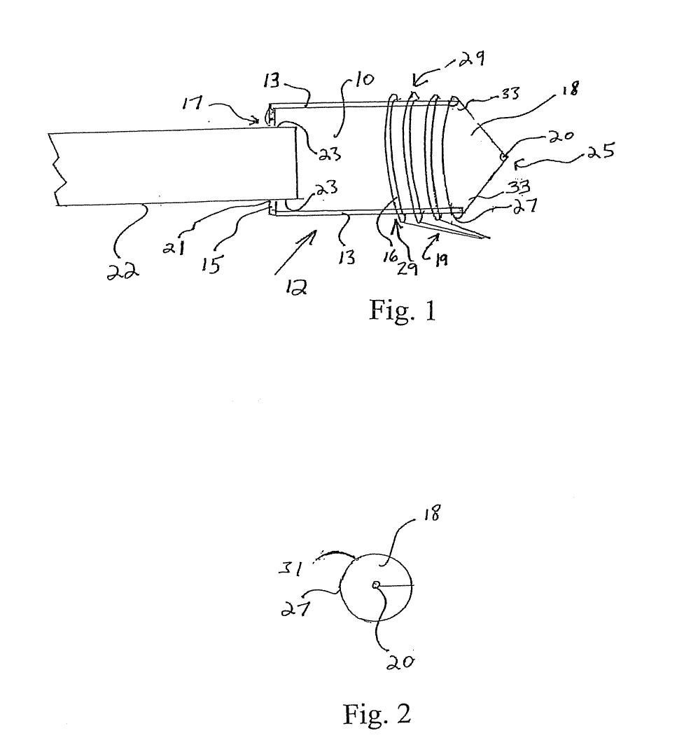

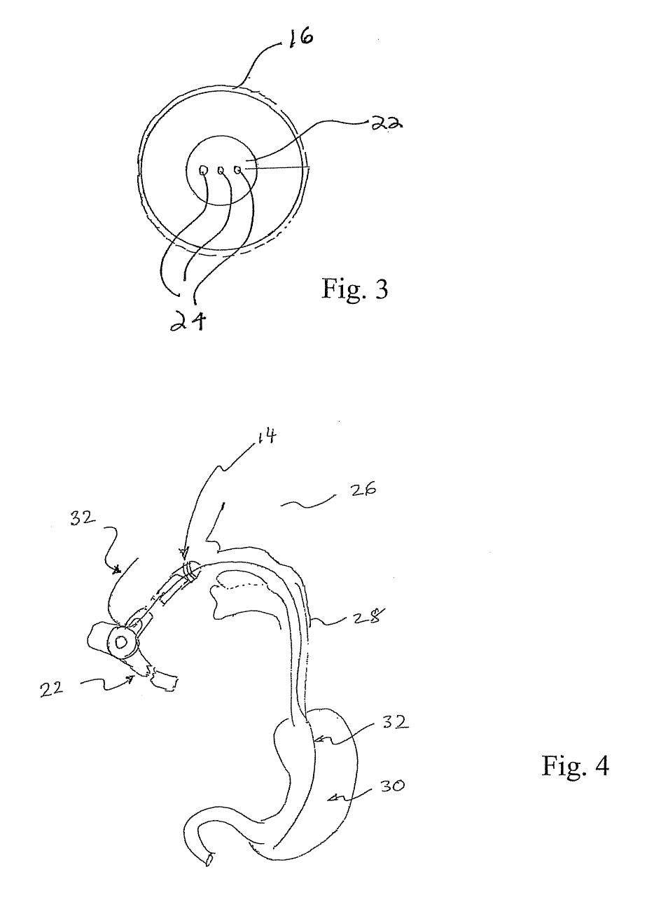

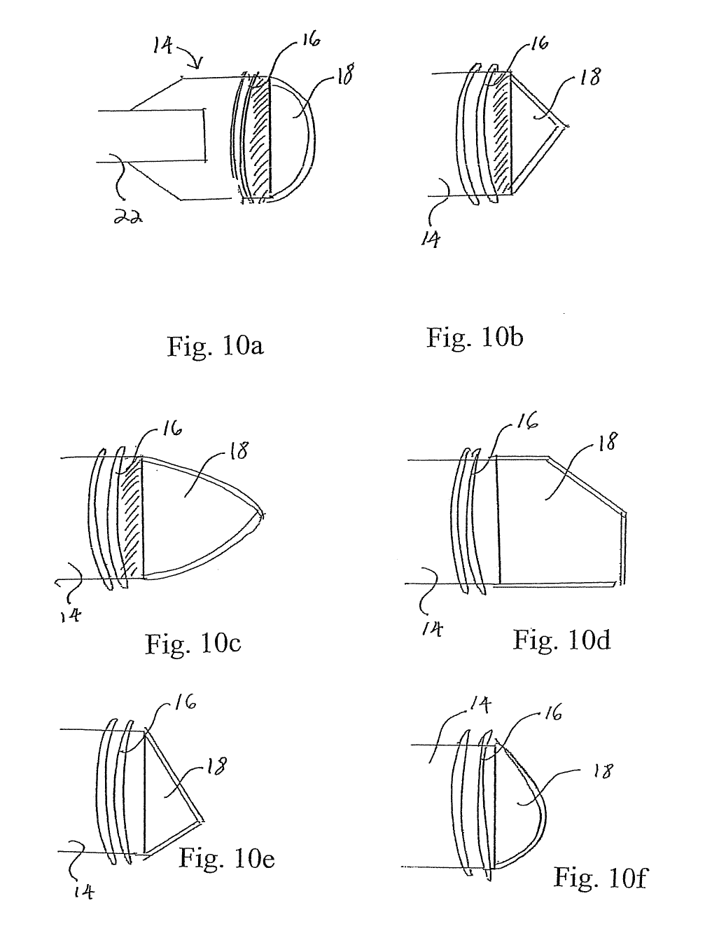

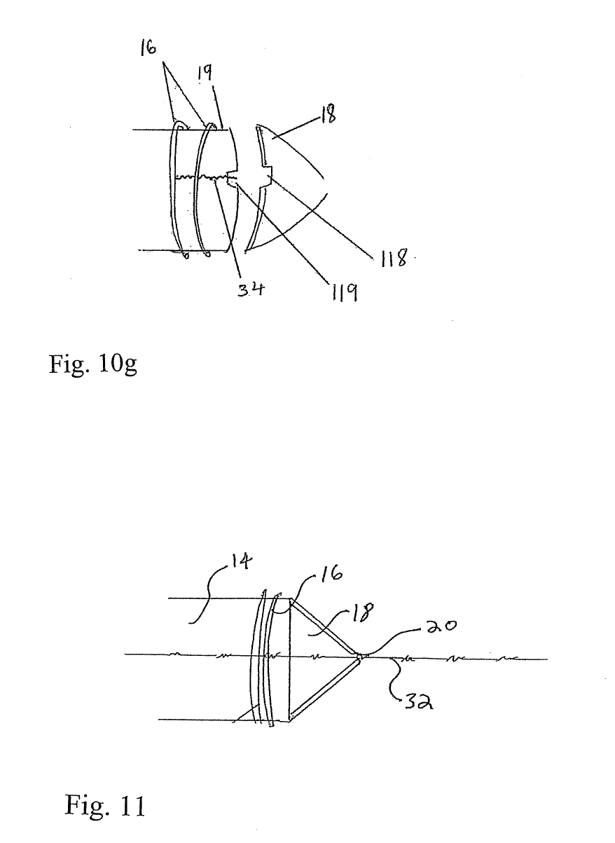

Referring to FIGS. 1-9, there is shown the device 12 in accordance with a preferred embodiment. The device 12 comprises a tubular main body or barrel 14 with a central cavity 10, a cap 18, and one or more elastic gastric bands 16. The cap 18 is comprised of an opening 20 through which a guidewire 32 may be threaded. The device 12 is further comprised of a triggering wire 34 detachably coupled to each band 16. Referring to FIG. 10g, in one embodiment, the cap 18 has one or more notches 118 or openings 118 structured and arranged to allow the unimpeded passage of the triggering wire 34. In this embodiment, the opening 118 is positioned at a portion of the rim of the cap 18 in such a manner as to allow the cap 18 to be removed without displacing the triggering wire 34 upon removal of the cap 18.

In the preferred embodiment, the barrel 14 is an elongated modified cylinder 14 comprising circular sidewalls 13, a base wall 15, a proximal end 17, and a distal end 19. In preferred embodiments, the device 12 has a length of 33-45 mm. The cylinder 14 is substantially hollow such that the central cavity 10 is formed by the walls 13, 15. The proximal end 17 diameter in preferred embodiments ranges from 9.85 mm to 10.85 mm. The distal end 19 diameter, at its largest point, is between 12 and 15 mm. Although the foregoing measurements accurately describe certain measurements of the device 12, the device need not have such dimensions. Rather, the device 12 may have other dimensions without departing from the scope and spirit of the invention. Referring to FIG. 10g, in one embodiment, the distal end of the cylinder 19 has one or more notches 119 or openings 119 structured and arranged to allow the unimpeded passage of the triggering wire 34. In this embodiment, the opening 119 is positioned at a portion of the rim of the cylinder 19 in such a manner as to allow the cap 18 to be removed without displacing the triggering wire 34 upon removal of the cap 18.

The proximal end 17 is adapted to be coupled with a conventional and commercially available endoscope 22. In the preferred embodiment an Olympus® GIF-H180 Evis Exera II endoscope with a diameter of 9.8 mm, a 2.8 mm channel, a 140 degree field of view, 103 cm working length, an angulation of 210/90 (up/down) and 100/100 (right/left) is used. However, the device is equally suitable for use with many other conventional medical devices. As may be seen in FIG. 1, the barrel's proximal end 17 comprises a base wall 15 with an attachment opening 21. The attachment opening 21 allows an endoscope 22 to be coupled to the barrel 14. In the preferred embodiment, the attachment opening 21 is generally circular and has an inside diameter slightly larger than an outside diameter of the endoscope 22. Attachment ridges 23 may be provided to enable the device 12 to remain securely fastened to the endoscope 22 during use. The base wall 15 may be angulated.

The distal end 19 of the barrel 14 is open and, as mentioned, may be notched 119 for passage of the triggering wire 34 and to correspond to the notch(es) 118 on the cap 18 embodiment shown in FIG. 10g discussed above. The openings 118,119 of this embodiment form a combined channel 118,119 when arranged in the manner suggested in FIG. 10g. This distal end 19 is structured and arranged to be capable of being removably coupled to the cap 18. In a preferred embodiment, the cap 18 is conical and hollow as shown in FIG. 1. However, the cap 18 need not be conical or hollow. In other embodiments, the cap 18 is spherical (FIG. 10a), conical (FIGS. 1 & 10b), elongated (FIG. 10c), beveled (FIG. 10d), angulated (FIG. 10e), or eccentric (FIG. 10f). It is preferred that the cap 18 be tapered such that a cap 18 distal end 25 has a smaller outside diameter than a cap proximal end 27. However, virtually any cap 18 configuration that allows a large caliber instrument 22 to be inserted into a patient's digestive tract may be used without departing from the spirit and scope of the present invention.

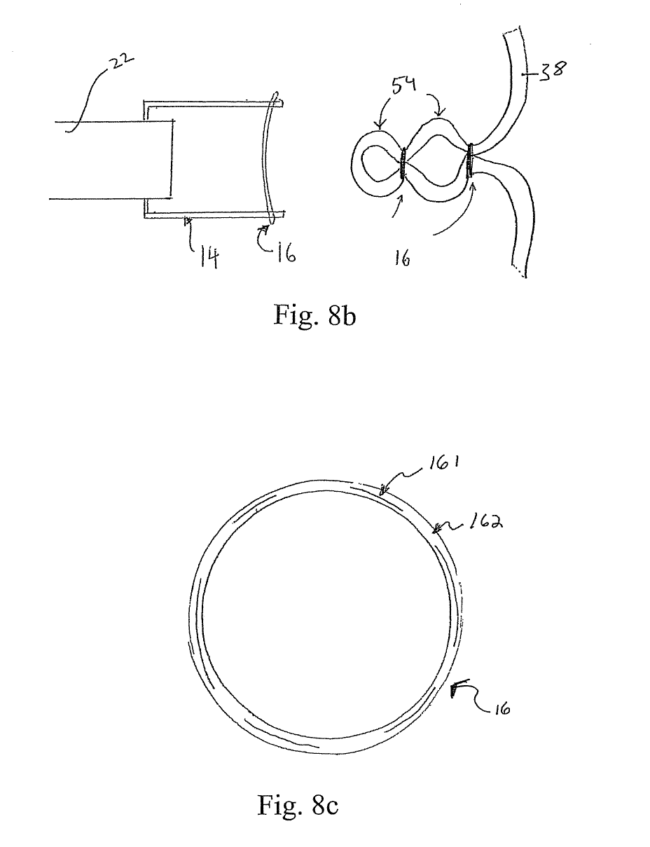

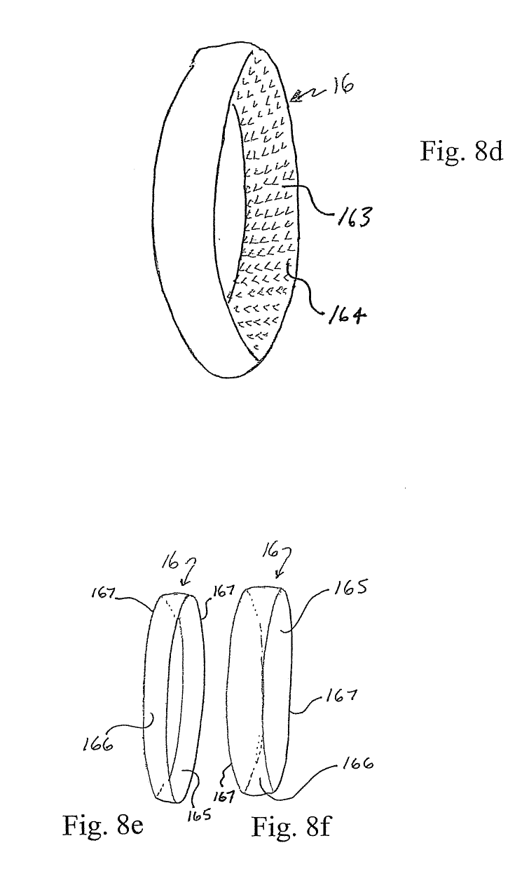

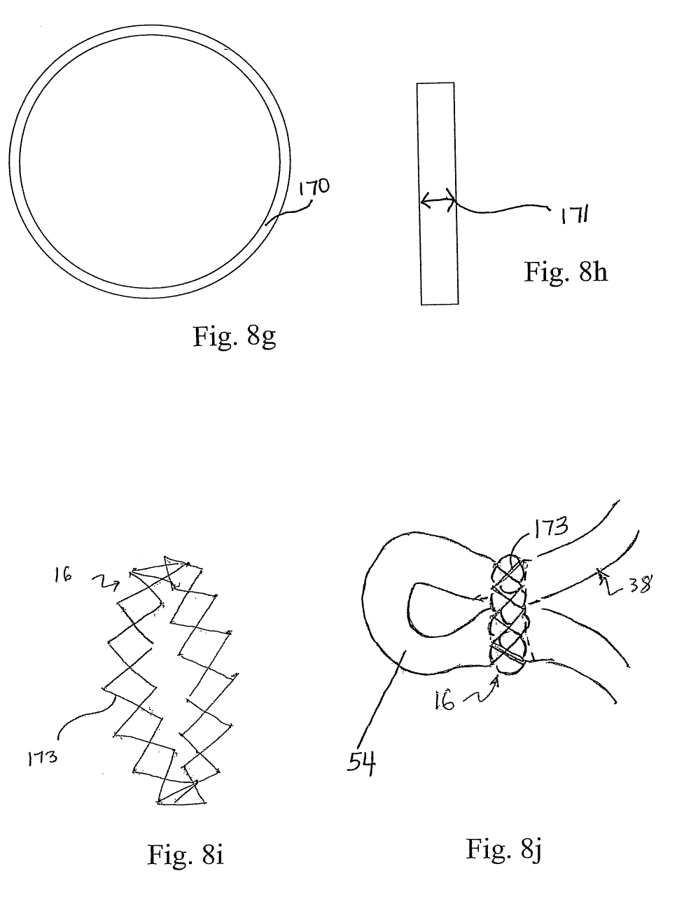

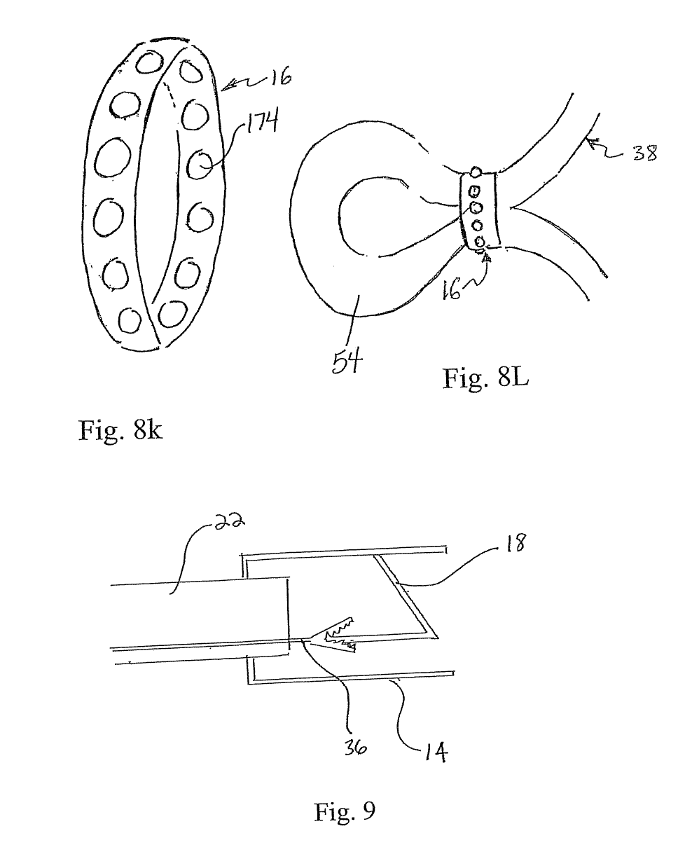

Referring to FIGS. 1, 2, and 10a-15, elastic bands 16 are disposed around a perimeter of the barrel 14. These bands 16 may be conventional gastric bands 16 used for tissue ligation. In the preferred embodiment, 6 to 10 bands 16 sold under the trade name Saeed Six-Shooter (Wilson-Cook) are used. Various embodiments of improved bands 16 which may be used are shown in FIGS. 8c-8f. In these embodiments, such bands 16 provide improved grip on the gastric wall. In FIG. 8c, there is shown a gastric band 16 comprising a central reinforcement portion 161. As shown, the band 16 is formed from an elastic material as in conventional gastric bands 16. However, this band 16 has an inner band 161 that is of conforming configuration to a circular outer band 162. This inner band 161 is formed from a resilient material such as metal and provides increased band 16 stability and greater strength than conventional bands 16.

Referring to FIG. 8d, a gastric band 16 in accordance with another embodiment is shown. In this embodiment, the band 16 comprises an inner ring surface 163 that is jagged or serrated. The serrated portion 163 of the band 16 comprises a series of points 164 or V shaped portions 164, which, when deployed about a portion of tissue 38, anchors the band 16 in a desired position. The band 16 of this embodiment may be formed from any suitable elastic material that permits the user to expand the band 16 around a portion of tissue 38 and contracts around the tissue 38 upon release. The band 16 may have an inner band 161 as shown in FIG. 8c or may be formed without such an inner band 161.

In other embodiments of an improved gastric band 16, the band 16 is an expanded surface band 16 as shown in FIGS. 8e and 8f. Referring to these figures, the bands 16 each have a conventional circular configuration, mesh or perforated structure, and each have an inner band surface 165, an outer band surface 166, and side surfaces 167. The inner and outer band surfaces 165,166 each have an unstretched length that comprises the width W and the side surface has an unstretched length comprising the thickness T. In the expanded surface band 16 shown in FIG. 8e, the Width W is approximately twice as wide as corresponding conventional band 16 surfaces. In this embodiment, and referring to FIGS. 8g and 8h, the thickness T is approximately equal to the width W.

In the expanded surface band shown in FIG. 8f, the unstretched width W is approximately three times as wide as corresponding conventional band 16 surfaces. The expanded width W permits a greater amount of inner band surface 167 area to contact gathered tissue and provides greater band 16 strength. In this embodiment, and referring to FIGS. 8g and 8h, the thickness T is approximately equal to the width W.

Although in the embodiment shown in FIGS. 8e and 8f the unstretched widths W and thicknesses T are equal, the widths W and thicknesses T need not be equal. Rather, the width W may be different a different length than the thickness T, but preferably the length of each is greater than or equal to 2 mm. For example, in one embodiment, the width W may be 3 mm while the thickness T is 2 mm. By way of another example, the thickness T may be 2 mm while the width W is 3 mm.

Still other embodiments of improved gastric bands 16 are shown in FIGS. 8i through 8L. Referring to FIG. 8i, the gastric band 16 of this embodiment comprises an elastic mesh structure. As shown in FIGS. 8i and 8j, the band 16 of this embodiment comprises a network of interwoven elastic strands 173 which collectively form the circular configuration of the band 16. In this embodiment, the band 16 may be expanded into the expanded position as shown in FIG. 8i. Upon release, the band 16 moves to a contracted or deployed position as shown in FIG. 8j.

Referring to FIGS. 8k and 8L, a band 16 having a perforated structure is shown. In this embodiment, the band 16 comprises a plurality of openings 174. These openings 174 are arranged such that the openings 174 are spaced along the inside surface of the band 16. In this embodiment, the openings 174 are through openings 174 such that the outside surface of the band 16 is in fluid communication with the inside surface. However, the openings 174 need not be through openings 174. Rather, the openings 174 can penetrate the inside surface of the band 16 without penetrating the outside surface. In FIG. 8L, the perforated band 16 is shown in a contracted position.

The mesh band shown in FIGS. 8i and 8j and the perforated bands shown in FIGS. 8k and 8L help reduce slippage of the band 16 once deployed about the gathered portion 54.

While the invention has been shown in connection with conventional gastric bands and improved gastric bands 16, virtually any surgical band 16 capable of permitting a surgeon to ligate tissue may be used.

To prepare the device 12 for use in surgery, the bands 16 are temporarily stretched to a diameter greater than an outside diameter of the barrel 14 and the bands 16 are placed in a position near the distal end 19 of the barrel 14 such that each band 16 generally encircles a central longitudinal axis of the barrel 14. The elastic nature of the bands 16 allows the bands 16 to remain elastically, but removably, coupled to the outside perimeter 29 of the barrel 14.

The cap 18 of the preferred embodiment is removably coupled to the distal end 19 of the barrel 14 such that the distal barrel opening 19 is covered. In the preferred embodiment, the cap 18 and barrel 14 have cooperative coupling elements 31, 33 that allow the cap 18 to remain in place during an insertion phase of the procedure and easily removed when the surgeon so desires. In the preferred embodiment, the cap 18 has a circular detent 31 that engages over a corresponding ridge 33 located at the outside of an extreme distal end 19 of the barrel 14. Although the cap 18 of the preferred embodiment has a diameter greater than the barrel 14 diameter at the respective coupling points, the barrel 14 and cap 18 may be structured differently without departing from the scope of the invention. For example, the cap 18 can have the same or smaller diameter than the barrel 14 at the coupling points 31,33 and other cooperative coupling mechanisms known to those skilled in the art may be used to allow the cap 18 to be removably coupled with the barrel 14.

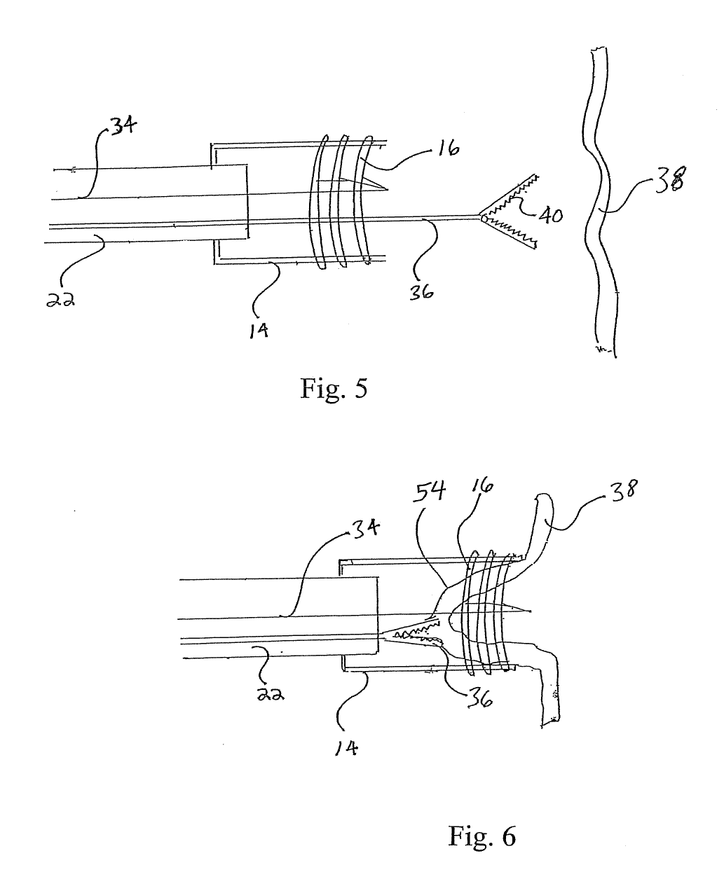

In one embodiment, the device 12 further comprises a band triggering wire 34. Referring to FIGS. 5 and 6, this wire 34 is arranged such that the surgeon can remotely remove a selected band 16 from the perimeter 29 of the barrel 14 when desired. The triggering wire 34 extends through the endoscope 22 and device cavity 10 and when pulled, displaces the distal most band 16 from the barrel end 19.

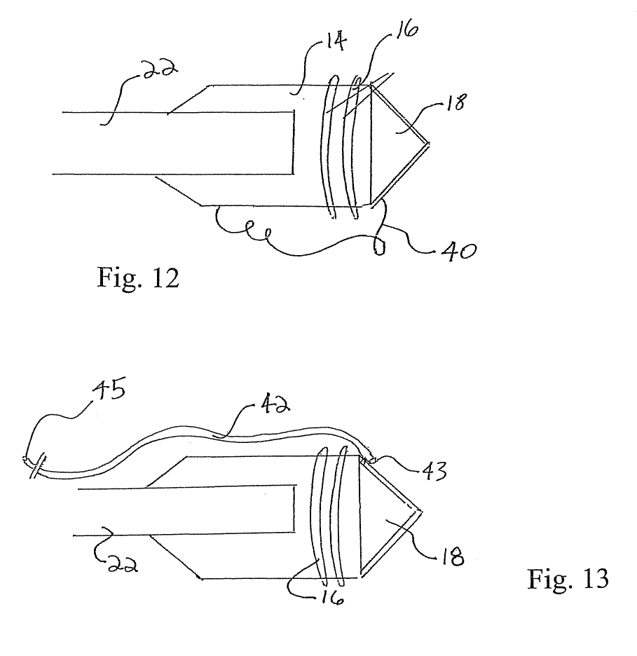

The device 12 further comprises a guidewire 32. The guidewire 32 is a conventional narrow gauge wire 32 used routinely by physicians to facilitate placement of medical devices such as catheters and stents. In the preferred embodiment, the guidewire 32 extends interiorly along the longitudinal axis of the endoscope 22, barrel 14, and cap 18 as shown in FIGS. 4 and 5. The cap 18 is comprised of an opening 20 through which the guidewire 32 may be threaded. However, the guidewire 32 need not be threaded through the interior of the endoscope 22, barrel 14 and cap 18. Rather, the guidewire 32 may be strung along the outside of the endoscope 22 and device 12, as shown in FIG. 12, such that the guidewire 32 passes through an external hook 48 or channel 48.

In a preferred embodiment, the surgeon inserts the guidewire 32 into the stomach 30 as shown in FIG. 4, and threads the device 12 over the guidewire 32 towards the patient's 26 stomach 30. Once the device 12 is introduced into the stomach 30, the surgeon may remotely remove the guidewire 32 and then remove the cap 18 with forceps 36. Referring to FIGS. 5 and 6, the surgeon, may dry the lining with a jet of air and then, using the same or different forceps 36, may grasp a portion of the stomach lining 38 so as to form a gathered portion 54. The surgeon may then retract this portion 54 into the cavity 10 of the device 12. In one embodiment, the forceps 36 comprises a snare 52 which may be used to excise the gathered portion 54.

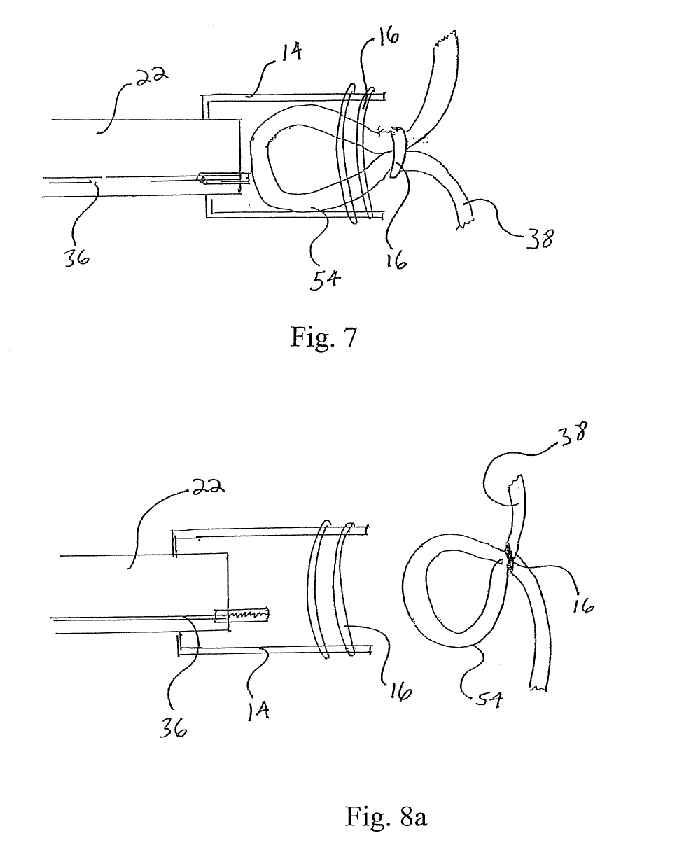

The surgeon then, using the triggering wire 34, displaces one of the bands 16 so that it contracts around the gathered portion 54 (FIGS. 7 and 8). Additional portions 54 of the gastric wall 38 may be banded in the same manner. Following the banding, the surgeon may desire to inject the gathered portions 54 with an agent, such as absolute alcohol, to ensure that the tissue is destroyed. Alternatively, after band ligation of the stomach tissue 38, the surgeon may elect to subject the gathered tissue 54 to thermal destruction using established techniques such as hot biopsy forceps cauterization and/or argon plasma coagulation. Upon completion of the procedure, the surgeon may grasp the cap 18, as shown in FIG. 9, and retract the endoscope 22 and device 12.

Following the placement of the bands 16 the patient is kept fasting and given intravenous medications to prevent gastric contractions for several hours.

The cap 18 permits the physician to easily insert the device 12. However, the device 12 may be easily removed without the cap 18. Therefore, the cap 18 may be left in the stomach 30 at the completion of the procedure. However, because leaving a plastic or indigestible cap 18 in the stomach 30 could have adverse effects on the patient 26, it is desirable to provide a cap 18 that, if left in the stomach 30, will not cause undue side effects to the patient 26. Therefore, in one embodiment, the cap 18 is formed from a digestible material such as cellulose or vegetable fiber. However, the device 12 need not be constructed from cellulose or vegetable fiber. Rather, for purposes of this embodiment, the cap 18 may be constructed of any suitable inert or digestible material that does not have to be retrieved. If the cap 18 is not to be left in the stomach 30, any suitable material, such as, metal, plastic, or synthetic matter, known in the art to be safe for use in endoscopic procedures may be used.

As stated, in a preferred embodiment, the device 12 and endoscope 22 are threaded by a guidewire 32. However, the device 12 need not be so threaded. Rather, the device 12 can be inserted visually such that the surgeon inserts the instrument 22 and device 12 by use of the endoscope's 22 camera. As a non-transparent cap 18 would obscure the endoscope 22 camera field of vision, in one embodiment of the present invention, the cap 18 is transparent. A transparent cap 18 need not be perforated with an opening 20 as the device 12 may be inserted visually as in conventional endoscopic procedures in which a guidewire 32 is not used. Once the device 12 is positioned in the desired location, the surgeon can displace the cap 18 as described above.

In a preferred embodiment, and as described above, the cap 18 is removably coupled to the barrel 14 and once displaced, may be either retrieved or left inside the patient. In another embodiment, and as shown in FIG. 12, the cap 18 may be attached to the barrel 14 or the endoscope 22 at a proximal location, by means of a tether 40. In this embodiment, the cap 18 remains remotely attached to the barrel 14 or the endoscope 22 after being removed from the barrel end 19. This tether 40 is a thin wire 40 or string 40 that is attached to the barrel 14 or the endoscope 22 at a point that is away from the distal end 19 so as to maintain separation between the tether 40 and the bands 16. The tether 40 may be of variable length, allowing the cap 18 to fall out of the way so as not to interfere with the band ligation procedure but so as to allow the cap 18 to be withdrawn with the rest of the assembly 12, 22 upon completion of the procedure, rather than grasped with the forceps 36, or snare 52 for removal.

As the cap 18 together with the endoscope 22 and device 12 are of a relatively large diameter, the surgeon may encounter some resistance when attempting to insert the assembly 12, 22 into the upper alimentary canal. Therefore, the surgeon may find it desirable and advantageous to lubricate the cap 18 during the insertion process. As depicted in FIG. 13, in one embodiment, the cap 18 is connected externally to the barrel portion 14 by means of a small diameter hollow tube 42. This tube 42 has a length that allows a tube distal end 43 to be inserted along with the assembly 12, 22 into the patient and a tube proximal end 45 to remain outside the patient. The tube 42 may be used to flush a fluid, such as water, over the cap's 18 surface. This flushing action may be used to remove debris and lubricate the tip of the assembly 22, 12. Further, the tube 42 has the additional advantage of being coupled to the cap 18 so that, for example, upon completion of the procedure, the surgeon may use this tube 42 to withdraw the cap 18.

Although in a preferred embodiment, the device 12 comprises a cap 18, the device 12 need not have a cap 18. Instead, referring to FIG. 15, the device 12 may be comprised of a balloon 44. In this embodiment, the balloon 44 occupies the cavity 10 and, upon inflation, presents a soft, pliable, and generally tapered surface extending beyond the barrel distal end 19 so as to allow the physician to overcome the resistance likely to be encountered in the process of insertion. The balloon 44 may be inflated prior to insertion and deflated once the device 12 is in the desired position. Alternatively, the balloon 44 may be used in conjunction with the cap 18 such that the balloon 44 may be used to displace the cap 18. In such an embodiment, the balloon 44 would be inflated remotely after the device 12 is placed in the desired position. The inflation action causes a portion of the balloon 44 to exert outward force on the cap 18 which is greater than the resistance force provided by the cap 18 and barrel 14 coupling mechanism 31, 33.

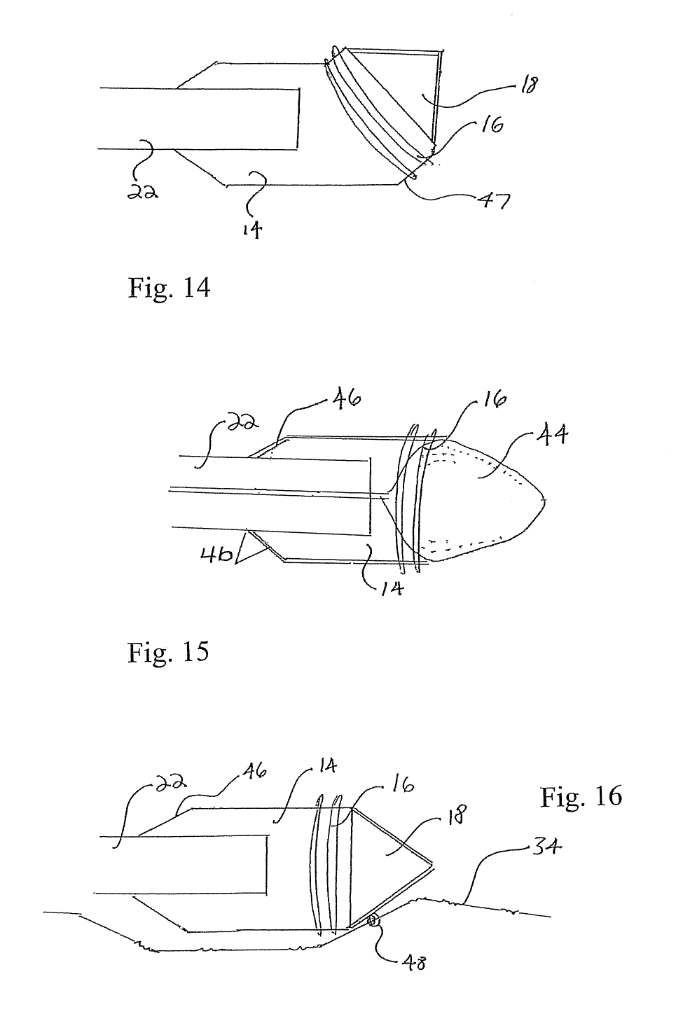

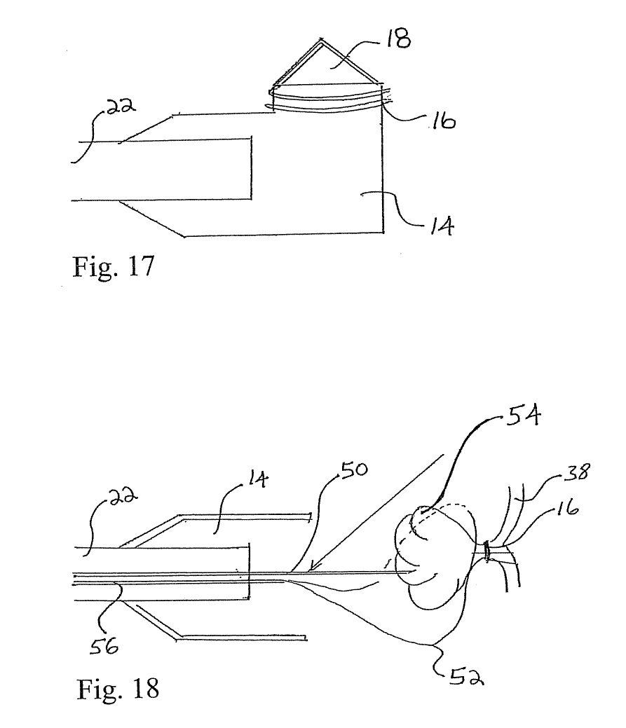

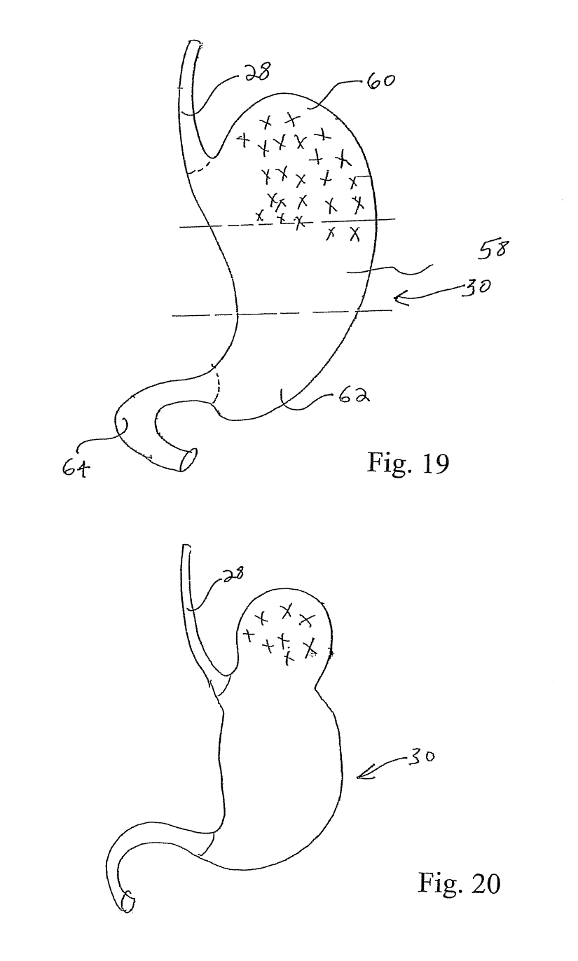

The distal end 19 of the device 12 can have other configurations. For example, and as depicted in FIG. 17, in one embodiment of the present invention, the opening 19 of the barrel 14 at the distal end 19 is vertical. In another embodiment and as shown in FIG. 14, the distal end 19 has a distal bevel 47 so as to create an angular distal 19 configuration. In this embodiment, the cap 18, is placed on a lateral aspect of the barrel 14, and may be displaced with a jet of water or air from the endoscope 22. The tissue 38 may be suctioned into the apparatus 12 using conventional vacuum suction techniques.

The proximal end 17 of the device 12 can have other configurations. For example, and as depicted in FIGS. 14-18, in some embodiments, the proximal end 17 has a proximal bevel 46 so as to create an angular distal 19 configuration. In these embodiments, the proximal end 17 is slanted or sloped so as to facilitate withdrawal of the assembly 12, 22 from the stomach 30 and esophagus 28 after completion of the procedure.

Also provided is a method for performing band ligation comprising the steps of: providing a device 12 comprising a generally tubular main body or barrel 14, a conical, spherical or tubular cap 18, one or more gastric bands 16, and a band triggering wire 34; inserting a guidewire 32 first end through a patient's mouth, through the esophageal opening, and into the patient's stomach 30, the stomach 30 having a gastric wall 38; introducing the device 12 over a second end of the guidewire through the mouth and esophageal opening and into the stomach 30; removing the cap 18 with endoscopic forceps 36, 52; targeting and preparing the selected areas by drying; withdrawing a full thickness of the gastric wall 38 into the device 12; and displacing one of said bands 16 such that said band 16 circumscribes a portion of said wall 38; wherein said guidewire 32 is threaded though the cap 18.

In one embodiment of the method of the present invention, the method comprises placing the greatest number of bands 16 at the top 60 of the stomach 30 with fewer bands 16 being placed lower down 58, 62, resulting in a pattern of an inverted triangle with its base above what would ordinarily be termed the apex. In this embodiment, the natural channel of the stomach 30 is maintained and the grinding function of the stomach 30 is preserved. By targeting this area specifically for reduction, the sense of satiety is stimulated rapidly, thereby reducing food intake. The fundus 60 and lateral capacitance areas of the stomach 30 would be reduced by following a pattern of targeted band placement and ligation. The procedure may have to be done once or repeatedly. However, other areas may be targeted in order to accelerate early satiety.

A hiatal hernia, a condition in which the fundus 60 of the stomach 30 herniates into the chest cavity through the diaphragm, is a common condition among obese patients. The method of the present invention may be used to treat this condition such that the size of the hiatal hernia (FIG. 20) is reduced.

In one embodiment, the method of the present invention is used to treat hyperacidity, acid reflux and Barrett's esophagitis, a precancerous condition of the esophagus 28 caused by acid reflux. These conditions result from elevated amounts of stomach acid produced by the fundus 60 of the stomach 30. Therefore, reduction of the fundus 60 is beneficial in the treatment of these conditions.

Referring to FIG. 8b, in one embodiment, two or more bands 16 are placed on the gathered tissue 54. Using two or more bands 16 increases a surgeon's ability to destroy the gathered tissue 54.

While there has been illustrated and described what is, at present, considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of this disclosure.

I claim:

1. An apparatus for introducing a medical instrument into a patient, the apparatus comprising:
a main body portion and a distal tapered portion, said apparatus having a cavity adapted to receive said instrument, said apparatus being further adapted to receive a guidewire.

2. The apparatus of claim 1 wherein said distal tapered portion is removably coupled to said main body.

3. The apparatus of claim 1 wherein said medical instrument is an endoscope.

4. The apparatus of claim 2 wherein said distal tapered portion comprises a through opening adapted to allow said guidewire to be threaded.

5. The apparatus of claim 2 wherein said distal tapered portion is retrievable.

6. The apparatus of claim 2 wherein said distal tapered portion is at least partially formed from biodegradable material.

7. The apparatus of claim 2 wherein said distal tapered portion is at least partially formed from inert material.

8. The apparatus of claim 1 further comprising a guidewire channel through which the guidewire may be threaded.

9. The apparatus of claim 8 wherein said guidewire channel is positioned on an external surface of said main body.

10. The apparatus of claim 1 wherein said apparatus further comprises one or more elastic bands elastically coupled to and disposed around a perimeter of said main body portion.

11. The apparatus of claim 10 further comprising a triggering wire adapted to displace said one or more elastic bands from said main body portion.

12. The apparatus of claim 11 wherein said cap further comprises one or more triggering wire channels through which said band triggering wire may be threaded.

13. The apparatus of claim 11 wherein said main body further comprises one or more triggering wire channels through which said band triggering wire may be threaded.

14. The apparatus of claims 11 wherein said cap further comprises one or more triggering wire channels through which said band triggering wire may be threaded, wherein said main body further comprises one or more triggering wire channels through which said band triggering wire may be threaded, and wherein at least one of said cap triggering wire channels and at least one of said main body triggering wire channels may be arranged to form a combined triggering wire channel.

15. The apparatus of claim 1 wherein said main body portion comprises a tapered proximal portion.

16. The apparatus of claim 1 wherein said distal tapered portion is inflatable.

17. The apparatus of claim 1 wherein said main body portion comprises an angled distal portion.

18. The apparatus of claim 2 wherein said distal tapered portion is at least partially transparent.

19. The apparatus of claim 2 wherein said tapered portion is tethered to said main body.

20. The apparatus of claim 5 further comprising a flush tube.

21. The apparatus of claim 20 wherein said flush tube is adapted for use in retrieving said distal tapered portion.

22. The apparatus of claim 1 further comprising a distal main body opening in a side wall.

23. A method for performing band ligation comprising the steps of:
providing a device comprising a main body portion, a tapered distal portion, one or more gastric bands, and a band triggering wire, said tapered distal portion being removably coupled to said main body portion;
introducing the device through the mouth and esophageal opening and into the stomach, the stomach having a gastric wall;
dislodging the distal tapered portion from said main body portion;
withdrawing a portion of the gastric wall into the device to form a gathered portion; and
displacing one or more of said bands such that said band circumscribes said gathered portion.

24. The method for performing band ligation of claim 23 further comprising the step of drying a portion of the gastric wall.

25. The method for performing band ligation of claim 23 further comprising administering intravenous medication to prevent contractions of the stomach.

26. The method for performing band ligation of claim 23 further comprising the step of retrieving said distal tapered portion.

27. The method for performing band ligation of claim 23 further comprising the step of injecting said gathered portion with a destruction agent.

28. The method for performing band ligation of claim 23 further comprising the step of thermally destroying said gathered portion.

29. The method for performing band ligation of claim 23 wherein said method is used to treat a hiatal hernia.

30. The method for performing band ligation of claim 23 wherein said method is used to treat hyperacidity.

31. The method for performing band ligation of claim 23 wherein said method is used to treat acid reflux.

32. The method for performing band ligation of claim 23 wherein said method is used to treat Barrett's Esophagitis.

33. The method for performing band ligation of claim 23 wherein a first plurality of gathered portions are gathered from a fundus area of the stomach and a second plurality of gathered portions are gathered from stomach areas more distal from said mouth than said fundus area, wherein the number of said gathered portions comprising said first plurality is greater than the number of said gathered portions comprising said second plurality.

34. The method for performing band ligation of claim 23 wherein a plurality of said gathered portions is arranged substantially in a ring pattern.

35. The method for performing band ligation of claim 23 wherein a plurality of said gathered portions is arranged substantially in a linear pattern.

36. A method for performing band ligation comprising the steps of:
providing a device comprising a main body portion, a tapered distal portion, one or more gastric bands, and a band triggering wire, said tapered distal portion being removably coupled to said main body portion;
providing a guidewire having first and second ends;
inserting the guidewire first end through a patient's mouth, through an esophageal opening, and into a patient's stomach, the stomach having a gastric wall;
introducing the device over the guidewire second end through the mouth and esophageal opening and into the stomach;
dislodging the distal tapered portion from said main body portion;
withdrawing a full thickness of the gastric wall into the device; and
displacing one or more of said bands such that said band circumscribes a portion of said wall.

37. The method for performing band ligation of claim 36 further comprising the step of drying a portion of the gastric wall.

38. The method for performing band ligation of claim 36 further comprising the step of administering medication to prevent contractions of the stomach.

39. The method for performing band ligation of claim 36 further comprising the step of threading said guidewire though a distal tapered portion through opening.

40. The method for performing band ligation of claim 36 further comprising the step of retrieving said distal tapered portion.

41. The method for performing band ligation of claim 36 further comprising the step of injecting said gathered portion with a destruction agent.

42. The method for performing band ligation of claim 36 further comprising the step of thermally destroying said gathered portion.

43. The method for performing band ligation of claim 36 wherein said method is used to treat a hiatal hernia.

44. The method for performing band ligation of claim 36 wherein said method is used to treat hyperacidity.

45. The method for performing band ligation of claim 36 wherein said method is used to treat acid reflux.

46. The method for performing band ligation of claim 36 wherein said method is used to treat Barrett's Esophagitis.

47. The method for performing band ligation of claim 36 wherein a first plurality of gathered portions are gathered from a fundus area of the stomach and a second plurality of gathered portions are gathered from stomach areas more distal from said mouth than said fundus area, wherein the number of said gathered portions comprising said first plurality is greater than the number of said gathered portions comprising said second plurality.

48. The method for performing band ligation of claim 36 wherein a plurality of said gathered portions is arranged substantially in a ring pattern.

49. The method for performing band ligation of claim 36 wherein a plurality of said gathered portions is arranged substantially in a linear pattern.

50. An improved gastric band comprising:
a circular elastic outer band and a concentric inner band.

51. An improved gastric band comprising:
a circular main body comprising outer, inner, and side walls, wherein said inner wall comprises a plurality of gripping points.

52. An improved gastric band comprising:
a circular body comprising outer, inner, and side walls, said band having a width defined by said side walls, and a thickness defined by said outer and inner walls; wherein said width is approximately equal to that of said thickness.

53. An improved gastric band comprising:
a circular body comprising outer, inner, and side walls, said band having a width defined by said side walls and a thickness defined by said outer and inner walls, wherein said width is different than said thickness.

54. An improved gastric band comprising an elastic mesh network.

55. An improved gastric band comprising a plurality of openings positioned around an inside perimeter of said band.
